(19) United States
(12) Patent Application Publication
(10) Pub. No.: US 2025/0041773 A1
(43) Pub. Date: Feb. 6, 2025

(54) METHOD FOR PURIFYING ADIPOSE TISSUE AND ASSOCIATED PURIFIED ADIPOSE TISSUE

(71) Applicant: NEOSYAD, AIX-EN-PROVENCE (FR)

(72) Inventors: Régis ROCHE, LORGUES (FR); François CABAUD, EGUILLES (FR); Xavier NELISSEN, VILLERS SAINT SIMEON (BE)

(21) Appl. No.: 18/717,115

(22) PCT Filed: Oct. 24, 2022
(86) PCT No.: PCT/FR2022/052008

§ 371 Date: Jun. 6, 2024

(30) Foreign Application Priority Data
Dec. 8, 2021 (FR) FR2113138

(51) Int. Cl. B01D 33/11; A61L 27/36; B01D 33/60; B01D 37/00

(52) U.S. Cl. CPC B01D 33/11; A61L 27/3604; A61L 27/3691; B01D 33/60; B01D 37/00; A61L 2430/34

(57) ABSTRACT

The application concerns a method for purifying adipose tissue, comprising at least: addition of a washing liquid to an adipose tissue to be purified comprising contaminants and present in a treatment volume delimited by means of retention of the adipose tissue and filtration of contaminants, filtration of a fraction of the contaminants of the adipose tissue, contained in the washing liquid, by centrifugation of the contents of the treatment volume by rotating the retaining and filtering means, and discharge of the washing liquid containing the filtered fraction of contaminants.

### CROSS REFERENCE TO RELATED APPLICATIONS

This application claims priority to PCT/FR2022/052008 filed Oct. 24, 2022 which claims priority to FR2113138 filed on Dec. 8, 2021. The contents of each of which are included by reference herein.

### TECHNICAL FIELD

The present application concerns a method of purifying adipose tissue and an associated purified adipose tissue suitable for reintroduction into a patient's body. The application especially finds application for autologous adipose tissue transplantation procedures (“lipofilling”) with aesthetic and reconstructive aims. In particular, the application finds a use for breast surgery operations; however, the implementation of the application is not limited to this application.

### PRIOR ART

Autologous adipose tissue transplantation procedures are used in surgery, especially breast surgery, to shape the breast to give it a more natural appearance after deep inferior epigastric perforator (DIEP), latissimus dorsi flap reconstruction, or after placing a breast implant. These operations involve taking fat from a patient in a donor area and then reintroducing it into the area of interest of the patient's body. These operations currently do not have a reproducible rendering and may require several interventions due to the resorption of the introduced material (loss of volume encountered after the operation). In addition, the fat is prepared for its reintroduction by an operator, surgeon or surgical assistant, which generates a relatively high cost of implementing the operation and a risk of error or loss of reproducibility. It is therefore desirable to propose a technique which simplifies and makes existing operations more reliable.

The document US 2020/0054824 sought to propose an improvement by implementing a closed loop system in which the fat is harvested to be prepared with a view to its reintroduction by being mixed with a washing liquid in a gravity filtration device marketed under the commercial reference Revolve®, and then to be reintroduced into the patient's body using a centrifugal pump. However, it is possible to improve the quality of the reintroduced fat.

### DISCLOSURE

The present application aims to overcome the disadvantages of the prior art and concerns a method for purifying adipose tissue, comprising at least:
adding a washing liquid to an adipose tissue to be purified comprising contaminants and present in a treatment volume delimited by means of retention of the adipose tissue and filtration of the contaminants,
filtering a fraction of the contaminants of the adipose tissue contained in the washing liquid by centrifuging the contents of the treatment volume by rotating the retaining and filtering means, and
discharging the washing liquid containing the filtered fraction of contaminants.

In a characteristic manner, the application implements a purification of adipose tissue by filtration of the contaminants through the retaining and filtering means due to the rotation of these means. This advantageously optimizes the quality of purified adipose tissue by reducing the presence of interstitial fluid and eliminating the presence of a blending or mixing member that could affect the integrity of adipose cells as in the case of the device marketed under the commercial reference Revolve® implemented in US 2020/0054824. The adipose tissue thus purified has, in particular, a much lower resorption rate than that obtained with the purification solutions of the prior art. Contaminants can include at least one of oil, blood or water. The addition of the washing liquid makes it possible to recover a fraction of the contaminants from the adipose tissue, which fraction is then removed by filtration by means of centrifugation of the contents of the treatment volume. As will be recalled below, if a purer adipose tissue is desired, it is possible to supplement this sequence of addition of washing liquid, filtration and discharge with other treatments making it possible to eliminate an additional fraction of the contaminants. The application uses centrifugal assisted filtration in which the separation between adipose tissue and contaminants is facilitated by rotating the retaining and filtering means. The adipose tissue is subjected to centrifugal accelerations against the inner wall of the retaining and filtering means, allowing contaminants to be drained through the pores of these means while the adipose tissue is retained in the treatment volume. The solution according to the application also differs from centrifugation techniques in centrifuges where adipose tissue and contaminants are present in a completely closed container and which use very high accelerations, generally greater than 400 G, to achieve phase separation and can lead to damage to adipose tissue. Moreover, compared to these techniques, the purification according to the application makes it possible to eliminate the oil present, the reintroduction of which into the patient's body must be avoided because it can form oily cysts.

In an exemplary embodiment, the filtration of the fraction of contaminants contained in the washing liquid comprises a succession of phases of rotation of the retaining and filtering means separated by one or more phases of interruption of this rotation. Such a characteristic advantageously contributes to further improving the quality of the purified adipose tissue obtained insofar as intermittent rotation of the retaining and filtering means further reduces the risk of damaging the adipose tissue compared to continuous rotation.

In one embodiment, the filtration of the fraction of contaminants contained in the washing liquid is carried out while the washing liquid is added to the treatment volume.

Centrifugation while the washing liquid is introduced into the treatment volume makes it possible to mix all the materials present in this volume and avoid any risk of blockage of the filtering and retaining means by adipose tissue.

In an exemplary embodiment, the method further comprises, after the washing liquid has been discharged, filtration of an additional fraction of the contaminants of the adipose tissue by centrifuging the contents of the treatment volume by rotating the retaining and filtering means.

Such a characteristic advantageously contributes to further improving the quality of the purified adipose tissue obtained.

In particular, after the washing liquid has been discharged, it is possible to perform:
an additional addition of the washing liquid to the adipose tissue,
a filtration of the additional fraction of the contaminants contained in the washing liquid thus added, by centrifuging the contents of the treatment volume by rotating the retaining and filtering means, and
a discharge of the washing liquid thus added containing the additional fraction of the contaminants filtered.

Such a characteristic advantageously contributes to further improving the quality of the purified adipose tissue obtained by allowing the elimination of an additional fraction of the contaminants. Of course and if desired, the addition of the washing liquid, filtration and discharge of the washing liquid can be repeated in order to remove an additional fraction of the contaminants.

In an exemplary embodiment, before the addition of the washing liquid, the adipose tissue to be purified is introduced into the treatment volume and a preliminary filtration of a fraction of the contaminants present in an interstitial liquid of this introduced adipose tissue is carried out by centrifuging the contents of the treatment volume by rotating the retaining and filtering means.

Such a characteristic advantageously contributes to further improving the quality of the purified adipose tissue obtained, especially by making it possible to further reduce the quantity of contaminants.

The preliminary filtration can be carried out while the adipose tissue to be purified is introduced into the treatment volume.

Such a characteristic advantageously makes it possible to be able to accommodate a larger volume of adipose tissue in the treatment volume because of the elimination of water as the adipose tissue is introduced.

In particular, the preliminary filtration can comprise a succession of phases of rotation of the retaining and filtering means separated by one or more phases of interruption of this rotation.

Such a characteristic advantageously contributes to further reducing the quantity of contaminants in the purified adipose tissue obtained while further reducing the risk of damaging the adipose tissue compared to continuous rotation.

In one embodiment, the contaminants are accelerated to between 5 G and 40 G when the retaining and filtering means are rotated. These acceleration values can be imposed on the contaminants contained in the washing liquid and/or on the contaminants contained in the interstitial liquid during the preliminary filtration which can be carried out before the addition of the washing liquid.

Such a characteristic advantageously contributes to optimizing the elimination of liquids present in the adipose tissue, and especially interstitial water, and to further improving the quality of purified adipose tissue. Moreover, limiting acceleration to values much lower than those used in conventional centrifugation also contributes to obtaining adipose tissue of optimal quality and with a small quantity of residual interstitial fluid allowing it to be easily injected into the patient's body.

In particular, an acceleration imposed on the contaminants during the preliminary filtration is greater than an acceleration imposed on the contaminants during the filtration of the contaminants contained in the washing liquid.

Such a characteristic advantageously contributes to further reducing the interstitial water content in the purified adipose tissue obtained as well as to further reducing the quantity of contaminants.

The application also relates to purified adipose tissue comprising adipose cells and having a total liquid content comprised between 0.05 grams per millilitre of adipose tissue and 0.15 grams per millilitre of adipose tissue with an oil content of less than or equal to 0.075 grams per millilitre of tissue.

Such an adipose tissue differs from adipose tissue directly taken from the patient by its composition and can be obtained by the purification method described above. In particular, it has a limited resorption rate when it is injected back into the patient's body while being easily injectable. Limiting the amount of oil in the tissue prevents the creation of oily cysts after injection into the patient, which can represent one of the most undesirable side effects of autologous adipose tissue transplantation. According to one example, the total liquid content is comprised between 0.07 grams per millilitre of adipose tissue and 0.12 grams per millilitre of adipose tissue. According to one example, the oil content in the purified adipose tissue is less than or equal to 0.05 grams per millilitre of adipose tissue, for example less than or equal to 0.03 grams per millilitre of adipose tissue. This content can, for example, be comprised between 0.005 grams per millilitre of adipose tissue and 0.05 grams per millilitre of adipose tissue, for example between 0.005 grams per millilitre of adipose tissue and 0.03 grams per millilitre of adipose tissue.

The application also relates to a surgical device comprising at least one tank containing the purified adipose tissue as described above in communication with an introduction element capable of introducing the purified adipose tissue from the tank into the patient's body.

Such a surgical device can correspond to a device marketed under the reference LipoGrafter® by the company MTFBiologics, incorporating adipose tissue purified by the method according to the application and connected to a cannula capable of injecting this adipose tissue into the patient's body. The skilled person will recognize that other surgical devices can be used for reintroduction into the patient's body.

### BRIEF DESCRIPTION OF THE DRAWINGS

FIG. 1 is a flowchart showing different steps of an example of a method according to the application.

FIG. 2 is an exploded schematic view of an example of a purification device that can be used in the context of the application.

FIG. 3 is a schematic sectional view of the purification device of FIG. 2 once assembled.

FIG. 4 is a schematic sectional view showing the purification device of FIG. 3 after displacement of the bottom.

FIG. 5 schematically represents a possible evolution of the speed of rotation of the retaining and filtering means which can be used in the context of the application for the steps of filtering contaminants.

FIG. 6 schematically and partially represents a surgical device comprising purified adipose tissue according to the application and capable of allowing its reintroduction into the patient's body.

### DESCRIPTION OF THE EMBODIMENTS

The following describes an example of a method of purifying adipose tissue removed from the patient's body for reintroduction into the patient's body for an autologous adipose tissue transplantation procedure, for example for breast surgery or at other parts of the body.

Adipose tissue is removed from the patient's body by suction using a suction cannula. The suction cannula is connected to a purification device, an example of the structure of which will be described below, which is capable of purifying the adipose tissue removed by filtration of contaminants assisted by centrifugation. The purification device especially comprises means for retaining adipose tissue and filtering contaminants, for example in the form of a filtering side wall, as well as a rotational drive device capable of rotating these means. These means have pores with a size configured to allow contaminants to pass through, for example in the form of a liquid medium, and retain adipose tissue. During removal, the adipose tissue is introduced into the purification device and, more particularly, into a treatment volume delimited by the retaining and filtering means (step E10).

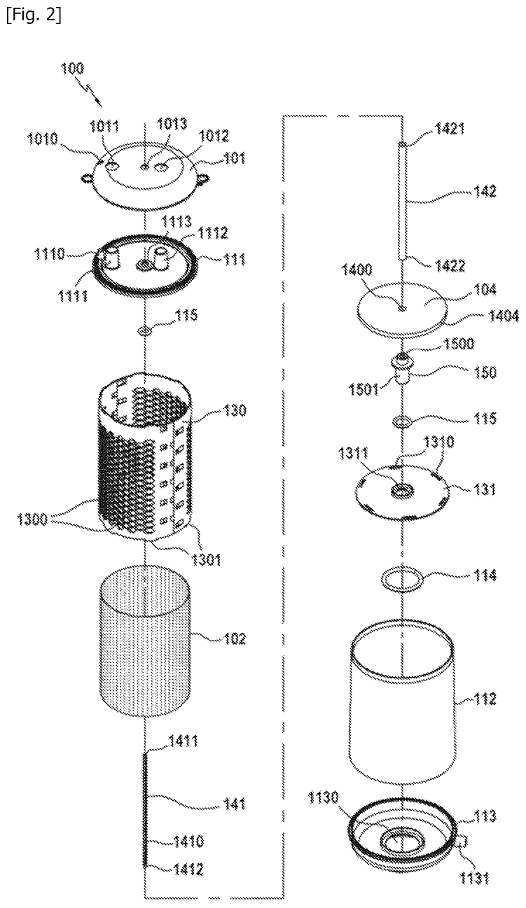

The following describes, with reference to FIGS. 2 to 4, an example of a purification device 100 that can be implemented within the scope of the application. This type of device can be used for all steps of filtration of contaminants carried out during the purification method. The device 100 is described by way of non-limiting illustration.

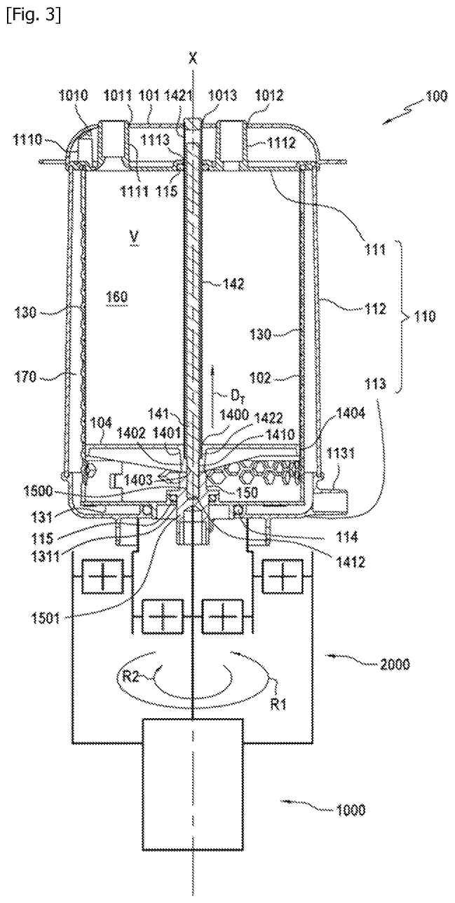

The purification device 100 comprises a sealed enclosure 110 formed here by a cap 111, a side wall 112 and a bottom 113. These elements are secured together in a sealed manner. The means 102 for retaining adipose tissue and filtering contaminants are present in the sealed enclosure 110. The means 102 delimit a centrifugation chamber 160 (visible in FIG. 3 and which defines the treatment volume V intended to receive the adipose tissue removed). In the example described here, the means 102 have a cylindrical shape, but the person skilled in the art will recognize that the means 102 can have other shapes suitable for centrifugation. The means 102 have a pore size configured to allow a liquid medium to pass through and retain adipose tissue. The pore size is chosen, in particular, to allow the passage of liquids such as oil, blood or physiological solution while retaining adipose tissue. In general, the pore size of the means 102 can be less than or equal to 1.5 mm, for example less than or equal to 0.5 mm. This size can be comprised between 0.05 mm and 1.5 mm, for example, between 0.2 mm and 0.5 mm. The means 102 can be made of a polymer material, for example polyester or polypropylene, but the person skilled in the art will recognize that other materials can be used.

In the example described here, a stiffening element 130 is present between the wall 112 of the sealed enclosure 110 and the means 102. Its function is especially to ensure the structural strength of the means 102 during centrifugation. The stiffening element 130 is, for example, made of a metal or plastic material and has a perforated structure defining a plurality of openings 1300 in order to allow the discharge of the liquid medium drained by the means 102.

The stiffening element 130 is associated with a rotary plate 131. More precisely, the stiffening element 130 comprises at its lower end teeth 1301 which cooperate with grooves 1310 present in the vicinity of the outer periphery of the rotary plate 131. The person skilled in the art will recognize that the element 130 can be connected to the plate 131 in different ways. The rotary plate 131 is connected to a rotary drive means which can be manual or motorized. In the example described here, the rotary plate is connected to an electric motor 1000, such as a stepper motor or a brushless DC motor, via a two-way clutch 2000 configured to drive the rotary plate 131 in rotation in a first direction of rotation R1 (FIG. 3).

Gaskets 114 and 115 are placed respectively below and above the rotary plate 131 in order to ensure sealing in the lower part of the purification device. Centrifugation is carried out by rotating the means 102. More precisely, the electric motor 1000 is controlled in the first direction of rotation R1 to drive the rotary plate 131 and the stiffening element 130 into engagement with the plate 131. The rotation of the plate 131 and of the stiffening element 130 causes the rotation of the means 102 which are of one piece with the stiffening element 130, for example by being glued or clipped to this element. The speed of the electric motor 1000 is controlled so as to apply a centrifugal force to the material in the volume V. Thus, an adipose tissue present in the volume V will be subjected to a centrifugal force against the inner wall of the means 102; this makes it possible to efficiently drain contaminants from the adipose tissue contained in the washing liquid or the interstitial fluid according to the purification step without damaging the adipose tissue.

During centrifugation, these contaminants passing through the means 102 and the stiffening element 130 are collected in a volume 170 delimited between the stiffening element 130 and the wall 112 of the enclosure 110. It is then possible to discharge the washing liquid or the interstitial liquid containing the contaminants via a discharge port 1131 present on the bottom 113 of the enclosure 110. In the example described here, the cap 111 comprises three ports 1110, 1111 and 1112 intended to be connected respectively to a device for suctioning adipose tissue (vacuum pump allowing removal), a conduit for the introduction of the adipose tissue removed into the volume V and a device for the delivery of the washing liquid. The purification device comprises a cover 101 comprising openings 1010, 1011 and 1012 which cooperate with the ports 1110, 1111 and 1112 of the cap 111. The vacuum pump connected to the purification device 100 which allows the removal of adipose tissue and its introduction into the volume V can also be connected to the discharge port 1131. The adipose tissue removed is retained in the device 100 by the means 102. The vacuum pump is also used to discharge contaminants, contained in the washing liquid or interstitial liquid, from the inside of the device 100 by suction. The suctioned materials are recovered in a waste container in communication with the vacuum pump and port 1131.

The purification device can also comprise a bottom 104 of the volume V, for example in the form of a movable collecting tray. As illustrated in the example of FIGS. 2 and 3, the collecting tray 104 is movable in translation in a direction D_{T} along the axis X of the means 102. More precisely, the purification device 100 comprises a threaded shaft 141 which extends vertically inside the centrifugation chamber 160 along the axis X of the means 102. The lower end 1412 of the threaded shaft is connected to the electric motor 1000 by means of a guide 150 and the two-way clutch 2000. The guide 150 comprises a housing 1500 in which the lower end 1412 is secured. A lower portion 1501 of the guide 150 is connected to a part of the two-way clutch 2000 which is engaged with the electric motor 1000 only when this motor transmits a rotational movement along a second direction of rotation R2 opposite to the first direction of rotation R1 used for the centrifugation. When the electric motor 10 rotates along the first direction of rotation R1, no rotational movement is transmitted by the two-way clutch 2000 to the guide 150 to which the threaded shaft 141 is connected and the means 102 are set in rotation due to of the rotation of the rotary plate 131. The contaminants contained in the washing liquid or the interstitial liquid are thus filtered through the means 102. When the motor 1000 rotates in the second direction of rotation R2, no rotational movement is transmitted by the two-way clutch 2000 to the rotary plate 131 and, consequently, to the means 102. The collecting tray 104 comprises, on its upper face, a central opening 1400 extended by a neck 1401 which extends from the lower face of the tray 104. The neck 1401 comprises a portion 1402 comprising a thread 1403 which cooperates with a thread 1411 of the threaded shaft 141. Thus, when the threaded shaft is driven in rotation in the second direction of rotation R2, the collecting tray 104 rises in the centrifugation chamber 160 in the direction D_{T}.

The peripheral edge 1404 of the collecting tray 104 is opposite the internal wall of the means 102. Thus, when the collecting tray 104 is moved in vertical translation in the direction D_{T}, this acts as a piston that scrapes the inner wall of the filter to collect more adipose tissue and facilitate the discharge of purified adipose tissue from the top of the device for introduction into the patient's body via a reintroduction device which will be described below in connection with FIG. 6. According to a particular aspect, the threaded shaft 141 can be housed in a protective sheath 142. The protective sheath 142 which extends between an upper end 1421 and a lower end 1422 makes it possible to prevent the threaded shaft 141 from coming into contact with the adipose tissue which, by accumulating at thread 1403 of the collecting tray 104, can block the displacement of this tray.

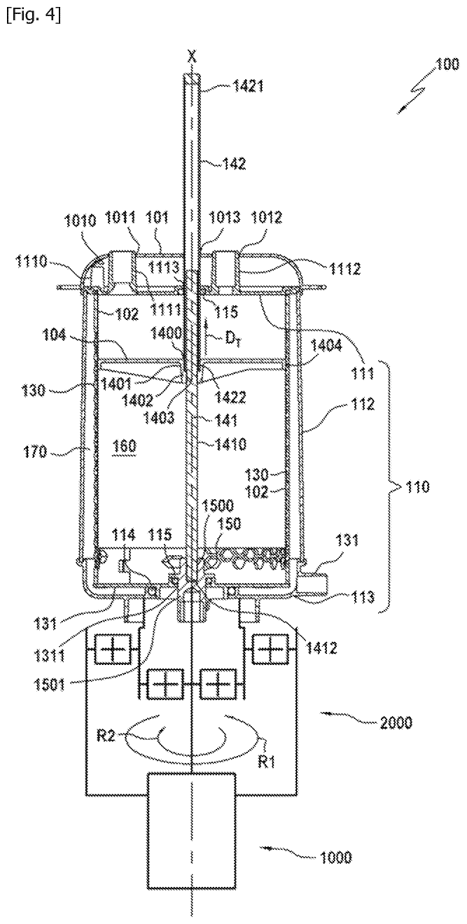

FIG. 4 shows the purification device 100 after actuation of the electric motor 1000 in the second direction of rotation R2 enabling the threaded shaft 141 to be driven in rotation. The rotation of the threaded shaft 141 causes the collecting tray 104 to move vertically in translation in the direction D_{T}.

The lower end 1422 of the protective sheath is secured in the opening 1400 of the collecting tray 104. In order to allow the movement of the protective sheath 142 during the displacement of the tray 104, the cap 111 and the cover 101, respectively, comprise an opening 1113 and an opening 1013 through which the sheath 142 slides. A gasket 115 is present around the opening 1113 in order to preserve the seal at the top of the centrifugation chamber.

In another exemplary embodiment, the means 102 are made of a rigid self-supporting material such as, for example, a metal material. In this case, the stiffening element 130 is no longer necessary and it is the self-supporting means 102 which are directly engaged with the rotary plate 131.

The following describes details on the filtration step of a fraction of the contaminants that are valid whatever the filtration step considered, especially for the preliminary filtration (step E20), as well as for the filtrations of steps E40, E60 and E90 which will be described below.

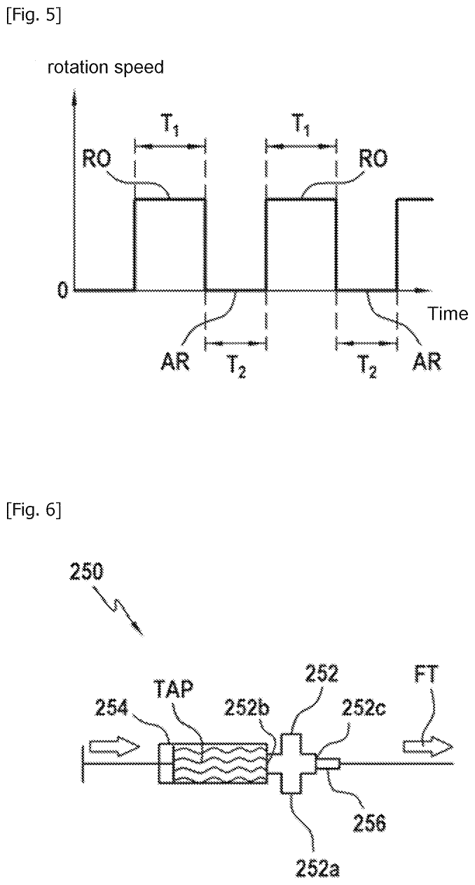

The filtration of a fraction of the contaminants, contained in the washing liquid or in the interstitial liquid, is carried out by centrifuging the contents of volume V by rotating the means 102. The means 102 are rotated around themselves. The means 102 are rotated around the axis X. The axis X can correspond to the axis of the height of the means 102, and for example, to an axis of symmetry or revolution of these means 102. When the means 102 are rotated, the adipose tissue and the contaminants can undergo an acceleration greater than or equal to 8 G, for example greater than or equal to 10 G or greater than or equal to 12 G. This acceleration measured in G corresponds to the ratio between the acceleration undergone by the material and the acceleration of Earth's gravity, which is approximately 9.81 m²/s. The acceleration experienced by the material corresponds to the ratio of the centrifugal force applied to the mass of the material concerned. The centrifugal force applied is equal to m*ω²*R where m is the mass of the object in question, ω is the angular velocity of the means 102 expressed in rad/s and R is the distance from the axis X of rotation to the centre of gravity of the object in question. During the rotation of the means 102, the adipose tissue and the contaminants can undergo an acceleration less than or equal to 40 G, for example less than or equal to 30 G, or less than or equal to 25 G or less than or equal to 20 G. This acceleration can be comprised between 8 G and 40 G or between 8 G and 30 G or between 8 G and 25 G or between 8 G and 20 G. This acceleration can be comprised between 10 G and 40 G or between 10 G and 30 G or between 10 G and 25 G or between 10 G and 20 G. This acceleration can be comprised between 12 G and 40 G or between 12 G and 30 G or between 12 G and 25 G or between 12 G and 20 G. The duration of a filtration step can typically be greater than or equal to 5 seconds. This duration can be less than or equal to 60 seconds. This duration can, for example, be comprised between 5 seconds to 60 seconds, for example between 15 seconds to 45 seconds. Regardless of the filtration step considered, the means 102 can be rotated intermittently, i.e., by successively imposing at least a first phase RO of rotation of the means 102, a phase AR of interruption of this rotation in which the means 102 are immobilized, and a second phase RO of rotation of the means 102, as illustrated in FIG. 5. The acceleration imposed on the contaminants during the first rotation phase RO can be the same or different from that imposed during the second rotation step RO. These accelerations can have the values in G described above. The phases RO of rotation of the means 102 can, according to one example, have a duration T1 greater than or equal to 5 seconds, for example comprised between 5 seconds and 15 seconds and the stop phases AR can, according to one example, have a duration T2 greater than or equal to 2 seconds, for example comprised between 2 seconds and 10 seconds. FIG. 5 shows rotation phases RO which have identical durations T1, as well as stop phases AR which also have the same duration T2, but it does not exceed the scope of the application if this is not the case. It does not exceed the scope of the application if more phases RO of rotation are carried out, thus a second phase AR of interruption of rotation can be carried out where the means 102 are immobilized after the second phase RO of rotation, then a third phase of rotation of the means 102 and so on. It is thus possible to carry out an alternation of phases of rotation of the means 102 and phases of interruption of this rotation during a step of filtering a fraction of the contaminants. It will be noted that the use of an intermittent rotation is especially advantageous for the preliminary filtration (step E20) and the filtration of the fraction(s) of the contaminants present in the washing liquid (step E60), but it would not exceed the scope of the application if all or part of the filtration steps are carried out by implementing a single phase of rotation of the means 102 applied continuously.

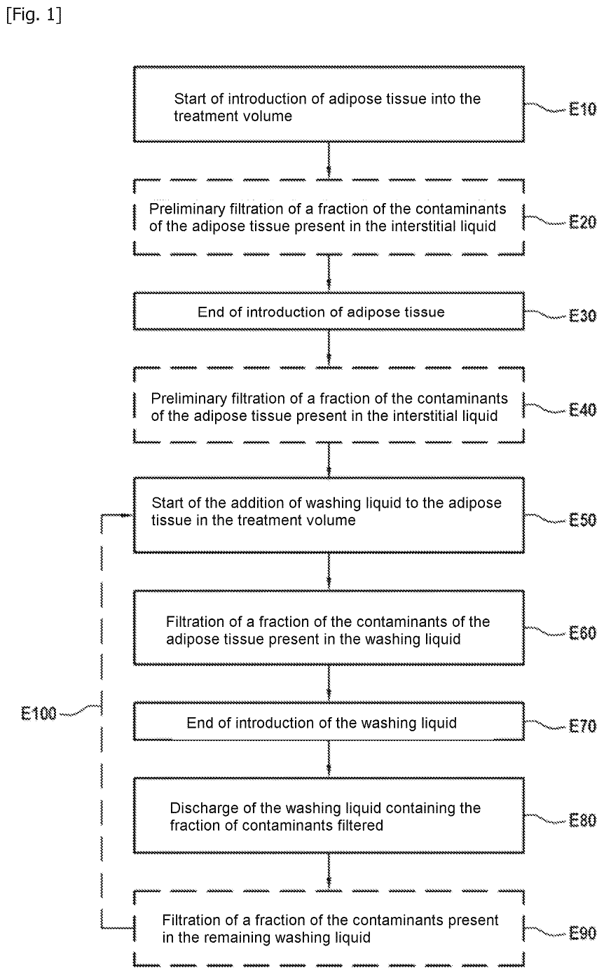

Step E20 of FIG. 1 concerns performing a preliminary filtration of a fraction of the contaminants contained in the interstitial liquid of the adipose tissue being introduced into the treatment volume V. Intermittent rotation of the means 102 as described above can be imposed during this step. Preliminary filtration removes contaminants in liquid form present in the introduced adipose tissue, such as blood, water or oil. During this phase, there is no introduction of a washing liquid into the volume V; only the adipose tissue removed is introduced into the volume V. Thus, it is possible to initiate the removal of the adipose tissue (step E10) then, when a first predetermined volume has been introduced into the volume V, to carry out a first phase RO of rotation of the means 102 while the removal of the adipose tissue continues, leading to the introduction of more adipose tissue into the volume V. According to one example, the first phase RO of rotation of the means 102 can be initiated when a volume comprised between 50 cm³ and 250 cm³ of adipose tissue has been introduced into the volume V. The means 102 are then stopped (phase AR) while adipose tissue removal continues during this phase AR of interruption after the first phase of rotation. When a second predetermined volume of adipose tissue has been introduced into the volume V, the second predetermined volume being greater than the first predetermined volume, the second phase RO of rotation of the means 102 can be initiated, and so on, if it is desired to continue the preliminary filtration. The example of FIG. 1 concerns the case where the preliminary filtration (step E20) is carried out while the adipose tissue is introduced into the volume V, but it does not exceed the scope of the application when the preliminary filtration is initiated only after completion of the adipose tissue removal.

Once the desired quantity of adipose tissue has been introduced into the volume V, the removal is stopped (step E30, stopping the introduction of adipose tissue into the volume V). The means 102 can then be rotated again so as to filter a fraction of the contaminants contained in the interstitial liquid (optional step E40), before the addition of the washing liquid (step E50) and as long as adipose tissue is no longer introduced into the volume V. A single continuous rotation of the means 102 can be carried out during step E40, but it would be possible, as a variant, to apply an intermittent rotation, as described above.

The vacuum pump used for removal is then activated so as to suction the interstitial liquid containing the filtered contaminants. The discharge time can be greater than or equal to 5 seconds, for example comprised between 5 seconds and 60 seconds.

The washing liquid is then introduced into the volume V (step E50). The washing liquid mixes with the removed adipose tissue to recover a fraction of the contaminants from this tissue. The washing liquid can thus recover blood, water and oil from the adipose tissue. The washing liquid can be a physiological solution. The washing liquid is introduced through the port 1112 in the example of device 100 described above. It will be noted that the washing liquid can be added while the means 102 are rotated as described above and, in particular, with intermittent rotation. The phases of rotation and interruption of rotation can be carried out during the introduction of the washing liquid (while it continues to be introduced into the volume V). The addition of the washing liquid can be carried out simultaneously with the filtration of the fraction of contaminants contained in the washing liquid (step E60). During this step E60, the washing liquid containing the fraction of contaminants passes through the pores of the means 102 to leave the volume V in which the adipose tissue is retained. The contaminants are recovered in the volume 170 in the example of the device 100 illustrated. The adipose tissue and the washing liquid containing the fraction of contaminants are thus separated. It will be noted that it would not exceed the scope of the application if the addition of the washing liquid and the filtration (step E60) are carried out sequentially, the filtration (step E60) being initiated once the addition of the washing liquid has been completed (step E70).

Once the introduction of the washing liquid has been completed (step E70), suction is carried out by the vacuum pump through the port 1131 in order to discharge the washing liquid containing the filtered fraction of contaminants from the device 100 and recover it in a waste container (step E80). This suction can be carried out, for example, using the vacuum pump used to remove adipose tissue. Discharge can be carried out for a duration greater than or equal to 5 seconds, for example comprised between 5 seconds and 60 seconds. If desired, it is then possible to continue to eliminate the remaining washing liquid containing contaminants by rotating the means 102 followed by suction in a manner similar to that described above (optional step E90) without adding washing liquid during this step. It is then possible, if desired, to repeat the steps of adding washing liquid (step E50), filtering a fraction of the contaminants present in the washing liquid (step E60) and discharging the washing liquid (step E80), as indicated by arrow E100 (optional).

Purification can also be completed after the sequences described above by compacting the adipose tissue in which the bottom 104 can be raised, such as a piston (see position of the bottom 104 in FIG. 4), and suction can be carried out on the compacted adipose tissue to discharge the air and any contaminants that may still be present.

The inventors conducted tests to measure the decrease in the quantity of oil and liquids in purified adipose tissue compared to adipose tissue directly removed from the patient's body. The results obtained over five test series are given in Tables 1 to 5 below.

TABLE 1
Series of test no. 1

| | Total | Quantity | Quantity | % | % |
|---|---|---|---|---|---|
| Initial | quantity | of oil | of liquid | Oil | Liquid |
| tissue 50 mL | (mL) | (g) | (g) | (g/mL) | (g/mL) |
| Tube No 1 | 50 | 5.33 | 7 | 11% | 14% |
| Tube No 2 | 50 | 5.44 | 7.5 | 11% | 15% |
| Tube No 3 | 50 | 5.1 | 7 | 10% | 14% |
| Initial sample | 50 | 5.29 | 7.17 | 11% | 14% |
| mean | | | | | |
| | Total | Quantity | Quantity | % | % |
| Purified | quantity | of oil | of liquid | Oil | Liquid |
| tissue 50 mL | (mL) | (g) | (g) | (g/mL) | (g/mL) |
| Tube No 1 | 50 | 1 | 5.5 | 2% | 11% |
| Tube No 2 | 50 | 0 | 5 | 0% | 10% |
| Tube No 3 | 50 | 1.1 | 3 | 2% | 6% |
| Tube No 4 | 50 | 0 | 4.2 | 0% | 8% |
| Tube No 5 | 50 | 0 | 3 | 0% | 7% |
| Treated sample | 50 | 0.42 | 4.14 | 1% | 8% |
| mean | | | | | |

TABLE 2
Series of test no. 2

| | Total | Quantity | Quantity | % | % |
|---|---|---|---|---|---|
| Initial | quantity | of oil | of liquid | Oil | Liquid |
| tissue 50 mL | (mL) | (g) | (g) | (g/mL) | (g/mL) |
| Tube No 1 | 50 | 6.1 | 10 | 12% | 20% |
| Tube No 2 | 50 | 6 | 11 | 12% | 22% |
| Tube No 3 | 50 | 5.9 | 10 | 12% | 20% |
| Initial sample | 50 | 6 | 10.33 | 12% | 21% |
| mean | | | | | |
| | Total | Quantity | Quantity | % | % |
| Purified | quantity | of oil | of liquid | Oil | Liquid |
| tissue 50 mL | (mL) | (g) | (g) | (g/mL) | (g/mL) |
| Tube No 1 | 50 | 2.4 | 7.5 | 5% | 15% |
| Tube No 2 | 50 | 2 | 7.5 | 4% | 15% |
| Tube No 3 | 50 | 2.5 | 7.5 | 5% | 15% |
| Tube No 4 | 50 | 2 | 5 | 4% | 10% |
| Tube No 5 | 50 | 2 | 5 | 4% | 10% |
| Treated sample | 50 | 2.18 | 6.5 | 4% | 13% |
| mean | | | | | |

TABLE 3
Series of test no. 3

| | Total | Quantity | Quantity | % | % |
|---|---|---|---|---|---|
| Initial | quantity | of oil | of liquid | Oil | Liquid |
| tissue 50 mL | (mL) | (g) | (g) | (g/mL) | (g/mL) |
| Tube No 1 | 50 | 4 | 19 | 8% | 38% |
| Tube No 2 | 50 | 4.1 | 20 | 8% | 40% |
| Tube No 3 | 50 | 4 | 20 | 8% | 40% |
| Tube No 4 | 50 | 5 | 20 | 10% | 40% |
| Initial sample | 50 | 4 | 19.67 | 8% | 39% |
| mean | | | | | |
| | Total | Quantity | Quantity | % | % |
| Purified | quantity | of oil | of liquid | Oil | Liquid |
| tissue 50 mL | (mL) | (g) | (g) | (g/mL) | (g/mL) |
| Tube No 1 | 50 | 1 | 7 | 2% | 14% |
| Tube No 2 | 50 | 1.2 | 7 | 2% | 14% |
| Tube No 3 | 50 | 0.9 | 7.2 | 2% | 14% |
| Tube No 4 | 50 | 0 | 5 | 0% | 10% |
| Tube No 5 | 50 | 0 | 5 | 0% | 10% |
| Treated sample | 50 | 0.62 | 6.24 | 1% | 12% |
| mean | | | | | |

TABLE 4
Series of test no. 4

| | Total | Quantity | Quantity | % | % |
|---|---|---|---|---|---|
| Initial | quantity | of oil | of liquid | Oil | Liquid |
| tissue 50 mL | (mL) | (g) | (g) | (g/mL) | (g/mL) |
| Tube No 1 | 50 | 0 | 19 | 0% | 38% |
| Tube No 2 | 50 | 0 | 20 | 0% | 40% |
| Tube No 3 | 50 | 0 | 20 | 0% | 40% |
| Tube No 4 | 50 | 0 | 20 | 0% | 40% |
| Initial sample | 50 | 0 | 19.67 | 0% | 39% |
| mean | | | | | |
| | Total | Quantity | Quantity | % | % |
| Purified | quantity | of oil | of liquid | Oil | Liquid |
| tissue 50 mL | (mL) | (g) | (g) | (g/mL) | (g/mL) |
| Tube No 1 | 50 | 0 | 7 | 0% | 14% |
| Tube No 2 | 50 | 0 | 7 | 0% | 14% |
| Tube No 3 | 50 | 0 | 7.2 | 0% | 14% |
| Tube No 4 | 50 | 0 | 5 | 0% | 10% |
| Tube No 5 | 50 | 0 | 5 | 0% | 10% |
| Treated sample | 50 | 0 | 6.24 | 0% | 12% |
| mean | | | | | |

TABLE 5
Series of test no. 5

| | Total | Quantity | Quantity | % | % |
|---|---|---|---|---|---|
| Initial | quantity | of oil | of liquid | Oil | Liquid |
| tissue 50 mL | (mL) | (g) | (g) | (g/mL) | (g/mL) |
| Tube No 1 | 50 | 1 | 12.5 | 2% | 25% |
| Tube No 2 | 50 | 2 | 10 | 4% | 20% |
| Tube No 3 | 50 | 1 | 10 | 2% | 20% |
| Initial sample | 50 | 1.3 | 10.83 | 3% | 22% |
| mean | | | | | |
| | Total | Quantity | Quantity | % | % |
| Purified | quantity | of oil | of liquid | Oil | Liquid |
| tissue 50 mL | (mL) | (g) | (g) | (g/mL) | (g/mL) |
| Tube No 1 | 50 | 0 | 5 | 0% | 10% |
| Tube No 2 | 50 | 0 | 3.1 | 0% | 6% |
| Tube No 3 | 50 | 0 | 3 | 0% | 6% |
| Tube No 4 | 50 | 0 | 3.4 | 0% | 7% |
| Treated sample | 50 | 0 | 3.625 | 0% | 7% |
| mean | | | | | |

The means over the five series of tests carried out are given in Table 6 below.

TABLE 6

| | Total | Quantity | Quantity | % | % |
|---|---|---|---|---|---|
| Initial | quantity | of oil | of liquid | Oil | Liquid |
| tissue 50 mL | (mL) | (g) | (g) | (g/mL) | (g/mL) |
| 17 tubes | 50 | 3.23 | 14.29 | 6.47% | 28.59% |
| | Total | Quantity | Quantity | % | % |
| Purified | quantity | of oil | of liquid | Oil | Liquid |
| tissue 50 mL | (mL) | (g) | (g) | (g/mL) | (g/mL) |
| 24 tubes | 50 | 0.67 | 5.42 | 1.34% | 10.84% |

The measurement method used to quantify the quantities of liquid and oil are provided below.

3 to 5×50 mL of adipose tissue are centrifuged at 1600 G (3000 rpm) in 50-mL graduated tubes for 3 minutes. At the outlet the tubes have 3 phases (from bottom to top):
the liquid phase (blood and infiltration liquid or washing liquid),
the adipose tissue phase, and
the oil phase.

For each tube, the oil is gently suctioned with a micropipette and a 1000 μL cone. The oil is deposited in a plastic cup to be weighed on a precision scale (0.001 g). For each tube, a 10-mL pipette connected to an electric pipettor is gently pushed into the bottom of each tube. All the liquid is suctioned, leaving only the adipose tissue phase in the tube. For each tube, the liquid is transferred into a receptacle and then weighed on a precision balance (0.001 g).

Once purification is complete, the surgeon reintroduces this tissue into the patient's body. In the example of device 100 shown in FIGS. 2 to 4, purified adipose tissue can be compacted against cap 111 in the device configuration of FIG. 4 and inserted into a surgical device, for example, such as, for example, a device marketed under the reference LipoGrafter® by the company MTFBiologics, in order to carry out the reintroduction into the patient's body. The introduction device can, for example, be connected to the port 1112 of the example of device 100 described above and the compaction pressure applied by the bottom 104 to the purified adipose tissue can be sufficient to allow the tissue to be transferred to the reintroduction device. As a variant, a pump can be disposed between the treatment volume V and the reintroduction device in order to induce this transfer. FIG. 6 diagrammatically and partially shows an example of a device 250 capable of allowing the purified adipose tissue TAP to be reintroduced into the patient's body. The device 250 comprises a body 252 provided with a first port 252a connected to the device 100 (connection not shown), a second port 252b connected to a syringe 254 and a third port 252c connected to an injection cannula 256. The purified adipose tissue is introduced from the treatment volume V into syringe 254 through the first port 252a. The first port 252a is provided with a check valve so as to allow the purified adipose tissue TAP to be injected into the patient through the cannula 256 by pressing the plunger of the syringe 254. The surgeon can act directly on the syringe 254 in order to induce the introduction of material into the patient's body, or in a variant, this action can be automated.

The expression “comprised between . . . and . . . ” should be understood to include the bounds.

1. A method for purifying adipose tissue, comprising:
addition of a washing liquid to an adipose tissue to be purified comprising contaminants and present in a treatment volume delimited by means of retention of the adipose tissue and filtration of the contaminants,
filtration of a fraction of the contaminants of the adipose tissue contained in the washing liquid by centrifuging the contents of the treatment volume by rotating the retaining and filtering means, and
a discharge of the washing liquid containing the filtered fraction of contaminants,
wherein a preliminary filtration of a fraction of the contaminants present in an interstitial liquid of the adipose tissue to be purified in the treatment volume is carried out before the addition of the washing liquid, by centrifuging the contents of the treatment volume by rotating the retaining and filtering means.

2. The method according to claim 1, wherein the filtration of the fraction of contaminants contained in the washing liquid comprises a succession of phases of rotation of the retaining and filtering means separated by one or more phases of interruption of this rotation.

3. The method according to claim 1, wherein the filtration of the fraction of contaminants contained in the washing liquid is carried out while the washing liquid is added to the treatment volume.

4. The method according to claim 1, wherein the method further comprises, after discharge of the washing liquid, filtration of an additional fraction of the contaminants of the adipose tissue by centrifuging the contents of the treatment volume by rotating the retaining and filtering means.

5. The method according to claim 4, wherein the following steps are carried out after the discharge of the washing liquid:
an additional addition of the washing liquid to the adipose tissue,
filtration of the additional fraction of the contaminants contained in the washing liquid thus added by centrifuging the contents of the treatment volume by rotating the retaining and filtering means, and
a discharge of the washing liquid thus added containing the additional fraction of the contaminants filtered.

6. The method according to claim 1, wherein the preliminary filtration comprises a succession of phases of rotation of the retaining and filtering means separated by one or more phases of interruption of this rotation.

7. The method according to claim 1, wherein the contaminants are accelerated to between 5 G and 40 G when the retaining and filtering means are rotated.

8. The method according to claim 1, wherein an acceleration imposed on the pollutants during the preliminary filtration is greater than an acceleration imposed on the pollutants during the filtration of the pollutants contained in the washing liquid.

9. A method for purifying adipose tissue, the method comprising:
adding a washing liquid to an adipose tissue to be purified comprising contaminants and present in a treatment volume;
filtering of a fraction of the contaminants of the adipose tissue contained in the washing liquid by centrifuging the contents of the treatment volume; and
discharging of the washing liquid containing the filtered fraction of contaminants, wherein a preliminary filtration of a fraction of the contaminants present in an interstitial liquid of the adipose tissue to be purified in the treatment volume is carried out before the addition of the washing liquid, by centrifuging the contents of the treatment volume.

10. The method according to claim 9, wherein the filtering of the fraction of the contaminants contained in the washing liquid comprises a succession of phases of rotation separated by one or more phases of interruption of the rotation.

11. The method according to claim 9, wherein the filtering of the fraction of contaminants contained in the washing liquid is carried out while the washing liquid is added to the treatment volume.

12. The method according to claim 9, wherein the method further comprises, after discharge of the washing liquid, filtering of an additional fraction of the contaminants of the adipose tissue by centrifuging the contents of the treatment volume.

13. The method according to claim 12, wherein the following steps are carried out after the discharge of the washing liquid:
adding additional washing liquid to the adipose tissue,
filtering an additional fraction of the contaminants contained in the washing liquid thus added by centrifuging the contents of the treatment volume; and
discharging the washing liquid thus added containing the additional fraction of the contaminants filtered.

14. The method according to claim 9, wherein the preliminary filtration comprises a succession of phases of rotation separated by one or more phases of interruption of the rotation.

15. The method according to claim 9, wherein the contaminants are accelerated to between 5 G and 40 G.

16. The method according to claim 9, wherein an acceleration imposed on the pollutants during the preliminary filtration is greater than an acceleration imposed on the pollutants during the filtration of the pollutants contained in the washing liquid.
